(19) United States
(12) Patent Application Publication
(10) Pub. No.: US 2023/0340699 A1
(43) Pub. Date: Oct. 26, 2023

(54) ENZYME COMPOSITION FOR CONVERTING PLANT BIOMASS INTO HIGH QUALITY TEXTILE GRADE FIBER

(71) Applicant: GENCREST PRIVATE LIMITED, Mumbai (IN)

(72) Inventors: Rashmi CHOWDHARY, Mumbal (IN); Anshika AGARWAL, Mumbai (IN); Nitin SHETYE, Mumbaiin (IN); Rohit BHARTI, Mumbai (IN); Netra GUPTE, Mumbai (IN)

(21) Appl. No.: 18/013,914

(22) PCT Filed: Jul. 2, 2021
(86) PCT No.: PCT/IB2021/055943

§ 371 Date: Dec. 30, 2022

(30) Foreign Application Priority Data
Jul. 2, 2020 (IN) 202021028275

(51) Int. Cl. C12N 9/24; C12N 9/02; C12N 9/42; C12N 9/26; D01C 1/02; C12N 9/20

(52) U.S. Cl. CPC D01C 1/02; C12N 9/0061; C12N 9/20; C12N 9/2414; C12N 9/2437; C12N 9/2482; C12N 9/2491; C12Y 110/03002; C12Y 301/01003; C12Y 302/01001; C12Y 302/01004; C12Y 302/01008; C12Y 302/01015; C12Y 302/01025

(57) ABSTRACT

The current invention discloses enzyme-based compositions for converting raw natural fibres from plant derived biomass into high quality textile grade fibres. The invention discloses at least one multi-component enzymatic formulation, and the optimal conditions for using these enzymatic formulations, which result in production of textile grade fibres from raw natural fibres. These textile grade fibres can be used in any industry, because of their high-quality parameters, and high spinnability index.

### FIELD OF INVENTION

The current invention relates to the field of obtaining textile grade fibres from plant derived biomass. The invention specifically relates a multi-enzyme formulation,and disclosesan enzymatic composition for processing raw natural fibres to obtain high quality fibres without the use of harsh chemicals.

### BACKGROUND

The demand for textile fibres is increasing constantly due to increasing world population, and the demand for fibres has almost doubled in the past 16 years or so. Along with providing food and clothing for the increasing world population, there is increasing need for conserving land andresources.Thus, efforts are being made to get fibres and food products from the same crops land resources. Moreover, synthetic petroleum-based fibres are non-biodegradable, hence present a different challenge to the ecosystem. These challenges can be met if high quality, textile grade fibres can be produced from various types of plant derived biomass, alot of which is also a by-product of the agricultural practices.

Globally, farming generates millions of tons of agricultural by-products each year. Some of the by-products are used as animal feed and for other small-scale applications.

In many countries, agricultural or agro-wastes are managed mainly by two methods. The first is the use of dry agro-residues as solid fuel in earthen/clay ovens or house stoves in the rural areas for cooking and producing heat. Huge amount of greenhouse gases such as CO₂, NOx, SOx, and particulate matter that can enter the respiratory system are produced during the burning of the lignocellulosic matter by this method of usage. This is neither an eco-friendly nor a healthy way of utilization of the agro-wastes. The second way of agro-waste management involves leaving the wet parts of the agro-waste openly in the site of cultivation for the amendment of soil by the fertilizer produced through the microbial decay of the biomass.This process, causes emission of another potent greenhouse gas, methane (CH₄) into the environment at a massive scale. Methane is produced during the microbial degradation of the agro-biomass by the native decaying microorganisms present in the soil. Another commonly practiced method for managing the enormous volume of straw and stubble is the open-field burning for mass destruction of the dry agro-waste. This is an environmentally detrimental process dueto the release of greenhouse gases and also due to destruction of top soil which harbors many beneficial micro-organisms.

Since many of the agricultural by-products contain substantial amounts of cellulose, especially in fibrous form, they can be used to produce fibres that can be used in various industries. But natural fibres which are extracted from plant biomass are generally coarser and have high lignin and hemicellulose content. This makes fibre very rigid and thick and hence finds limits its application to very few sectors such as handicrafts, handlooms, upholstery. The process of conversion of such thick and rigid fibres into more softer fibres involves chemical treatments which are very harsh and pose environmental concerns. Enzymes used so far in this area are very limited. The quality of the fibres achieved so far by using just enzymes is usually not sufficient for textile application due to difficulty in spinning it into yarn, and requires further harsh chemical steps to make it into spinnable grade, but with other drawbacks such as brittleness and loss in luster of thefibres.

Also considering the fact that textile industry is one of the most highly polluting industries globally, eco-friendly ways of converting the lignocellulosic waster from agricultural activities into textile grade fibres would lead to decreased pollution from the agricultural as well as textile industry. It is thus anurgent requirement to formulate eco-friendly compositions for making high quality textile grade fibres from cellulosic biomass without using harsh chemical or mechanical means. The current invention discloses an enzyme formulation that converts agricultural waste into high quality textile grade fibres by an entirely eco-friendly method. Moreover, the products made from these agricultural by-products by using these eco-friendly enzymatic compositions, are 100% bio-degradable with added qualities such as - breathability, hypoallergenicity, highhygroscopicity, and are easily spinnable into yarns. The current invention discloses an enzyme-based composition to enzymatically convert plant derived biomass into high quality textile grade fibres, which does not require strong chemical treatments, and also does not lead to loss of quality in the fibre thus produced.

### SUMMARY

One embodiment of the current invention is an enzyme formulation for converting raw natural fibres to textile grade fibres comprising enzymes selected from the group consisting of Cellulase, xylanase, pectinase, polygalacturonase, lipase, alpha amylase, mannanase and laccase.

In one embodiment, the enzyme formulation disclosed herein comprises 800-1000 U/ml Cellulase, 10,000-15000 U/ml xylanase, 100-300 U/ml pectinase, 100-200 U/ml, polygalacturonase, 500-700 U/ml lipase, 300-500 U/ml alpha amylase, 50-100 U/ml mannanase and 10-20 U/ml laccase .

In one embodiment, the enzyme formulation disclosed herein is added at a concentration of 0.5-1% to the fibre treatment bath for conversion of raw natural fibres to textile grade fibres. In one embodiment, the enzyme formulation disclosed hereinis stable at pH 4.5-5.5 and at temperature range of 20-50° C.

In one embodiment, the enzyme formulation disclosed hereinfurther comprises non-ionic surfactant and at least one stabilizer.

In one embodiment, the stabilizer is propylene glycol, glycerol, sugar or sugar alcohol.

In one embodiment, the textile grade fibres produced by treating the raw natural fibres by the enzyme formulation disclosed hereinare spinnable into yarn.

In one embodiment, the yarn produced from the textile grade fibres is woven by handloom or powerloom.

In one embodiment, the enzyme formulation disclosed hereinconverts raw fibres that have not been pre-treated by acid or alkali into textile grade fibres.

In one embodiment, the enzyme formulation disclosed hereinconverts raw natural fibres are from banana, hemp, nettle, flex, jute, pineapple, sisal, or remi plants.

In one embodiment, the textile grade fibres are maximum 75 dpf and breaking strength is at least 50 g.

In one embodiment, the current invention encompasses a method of making textile grade fibre from raw natural fibres, the method comprising the step of contacting the raw natural fibres with the enzyme formulation of claim 1 for 2-4 hours, wherein the enzyme formulation is present at a concentration of 0.5 - 1% in the fibre treatment bath.

### DETAILED DESCRIPTION

The current invention discloses an enzyme-based composition for production of textile grade fibres from plant derived biomass. The current invention particularly relates to an enzymatic formulation for making high quality textile grade fibre by processing raw natural cellulosic fibre from plant derived biomass byusing various enzyme activities along with mild chemical and mechanicaltreatment.

Cellulose is the most abundantly available organic matter on earth, is, in its natural and regenerated form a major source of fibre for textile industry. Plants are the major source of natural cellulosic fibres.

The current invention provides an enzymatic formulation for converting raw natural fibres from plant-based or plant derived biomass, into high grade textile fibres. This enzyme-based formulation is eco-friendly, and is used for production of textile fibres with high quality and high spinnability index, which can be used in any industry.

### Definitions

The term “plant derived biomass” as used herein is defined as biomass extracted from plants. It can be from plants that are specifically grown for obtaining that biomass, or can be a by-product of the main crop. Plant-derived biomass can be from any plant, including naturally growing plants, or agricultural crops.

The major constituent of the plant cell wall is “lignocelluloses”, which consists of lignin (15-20%), hemicellulose (25-30%) and cellulose (40-50%). These components together form a three-dimensional complex network bound by covalent and non-covalent interactions. Lignocellulose is generally found, for example, in the fibres, pulp, stems, leaves, hulls, canes, husks, and/or cobs of plants or fibres, leaves, branches, bark, and/or wood of trees and/or bushes. Examples of lignocellulosic materials include, but are not limited to, agricultural biomass, such as farming and/or forestry material and/or residues, branches, bushes, canes, forests, grains, grasses, short rotation woody crops, herbaceous crops, and/or leaves; crop residues, such as corn, millet, and/or soybeans, herbaceous material and/or crops; forests; fruits; flowers; needles; logs; roots; saplings; shrubs; switch grasses; vegetables; fruit peels; vines; wheat midlings; oat hulls; hard and soft woods; or any combination thereof.

Hemicelluloses consist of xylan, a heteropolysaccharide substituted with monosaccharides such as L-arabinose, D-galactose, D-mannoses and organic acids such as acetic acid, ferulic acid, glucuronic acid interwoven together with help of glycosidic and ester bonds. Depolymerization of this complex polymer is essential for its efficient utilization in different industrial application.

The term ″raw natural fibre″ as used herein is defined as the fibres which are mechanically extracted from plant biomass using manual process or machine extractors. Examples of raw natural fibres includes, without limitation, fibres derived from Banana, hemp, Bamboo, nettle, flex, ramie, jute, kenaf, sesal, abacca, coconut but not limited to these.

Raw fibres are derived from plant biomass by machine extractors, the machine strips the biomass open and extract the bast fibres. In some embodiments, no machine extraction is done to open the raw natural fibres before the enzymatic treatment.

The terms “enzymatic formulation”, “enzyme cocktail” and “enzymatic composition” are used interchangeably herein, and refer to a formulation comprising at least one enzyme, and it does not comprise any chemicals.

“Cellulase” or “cellulases”, as used herein, refer to an enzyme capable of hydrolyzing cellulose to glucose. Non-limiting examples of cellulases include mannan endo-1,4-β-mannosidase, 1,3-β-D-glucan glucanohydrolase, 1,3-β-glucan glucohydrolase, 1,3-1,4-β-D-glucan glucanohydrolase and 1,6-β-D-glucan glucanohydrolase.

“Xylanase” or “xylanases”, as used herein, refer to an enzyme capable of hydrolysing xylan to xylobiose and xylotriose.

Xylanase is a group of enzymes consisting of endo-1,4-β-d-xylanases (EC 3.2.1.8), β-d-xylosidases (E.C. 3.2.1.37), α-glucuronidase (EC 3.2.1.139) acetylxylan esterase (EC 3.1.1.72), α-1-arabinofuranosidases (E.C. 3.2.1.55), p-coumaric esterase (3.1.1.B10) and ferulic acid esterase (EC 3.1.1.73) involved in the depolymerization of xylan into simple monosaccharide and xylooligosaccharides.

The term “pectinase” includes any acid pectinase enzyme. Pectinases are a group of enzymes that hydrolyse glycosidic linkages of pectic substances mainly poly-1,4-alpha-D-galacturonide and its derivatives which enzyme is understood to include a mature protein or a precursor form thereof, or a functional fragment thereof, which essentially has the activity of the full-length enzyme. Furthermore, the term pectinase enzyme is intended to include homologues or analogues of such enzymes. Pectinases can be classified according to their preferential substrate, highly methyl-esterified pectin or low methyl-esterified pectin and polygalacturonic acid (pectate), and their reaction mechanism, beta-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by the Enzyme Nomenclature (1992) such as pectate lyase (EC 4.2.2.2), pectin lyase (EC 4.2.2.10), polygalacturonase (EC 3.2.1.15), exo-polygalacturonase (EC 3.2.1.67), exo-polygalacturonate lyase (EC 4.2.2.9) and exo-poly-alpha- galacturonosidase (EC 3.2.1.82).

Laccases, belong to the enzyme family of multi-copper oxidases (MCOs), are classified as benzenediol oxygen reductases (EC 1.10.3.2) and are also known as urushiol oxidases and p-diphenol oxidases. They are considered versatile enzymes capable of oxidizing a large number of phenolic and non-phenolic molecules due to their low substrate specificity, using oxygen as electron acceptor and generating water as a by-product.

Endo-1,4-β-d-mannanase (EC 3.2.1.78) catalyzes the random cleavage of β-d-1,4-mannopyranosyl linkages within the main chain of galactomannan, glucomannan, galactoglucomannan, and mannan. They liberate short-chain β-1,4-manno-oligomers, which can be further hydrolyzed to mannose by β-mannosidases (EC 3.2.1.25).

α Amylases are starch hydrolases. Amylases are responsible for hydrolysis of starch to oligosaccharides. α-Amylase hydrolyzes the 1,4-α-glucoside bonds in compounds involving three or more molecules of glucose. β-Amylase liberates (mainly) β-maltose from starch and other compounds.

In one embodiment, the amylases used in the current invention are alpha-amylases.

As used herein, enzyme activity is defined in units. 1 unit of enzyme (U, used interchangeably herein with IU or international units) is the amount of enzyme that catalyzes the reaction of 1 µmol of substrate per minute.

Activity definitions of enzymes used in the current invention are given below. The substrates used for activity assay can be any substrates known for the given enzymes. Moreover, the enzymes used can be from any of the known sources.

### Cellulase:

One unit of activity corresponded to the quantity of enzyme releasing 1umol of dextrose reducing sugar (in glucose equivalents) per minute per ml under the assay conditions. Any suitable substrate can be used for assessing the activity. The substrate used herein is Carboxymethylcellulose sodium salt -low viscosity (SIGMA- ALDRICH).

### Xylanase:

One unit of activity corresponds to the quantity of enzyme releasing lumol of xylose reducing sugar (in glucose equivalents) per minute per ml under the assay conditions. The substrate used herein for assessing xylanase activity is Xylan from beechwood (SRL).

### Pectinase:

One unit of activity corresponds to the quantity of enzyme releasing lumol of D-galacturonic acid reducing sugar (in glucose equivalents) per minute per ml under the assay conditions. The substrate used herein is Pectin from apple (SIGMA).

### Polygalacturonase:

One unit of activity corresponded to the quantity of enzyme releasing 1 µmol of D-galacturonic acid reducing sugar (in glucose equivalents) per minute per ml under the assay conditions. The substrate used for assessing the activity in the current invention is Polygalacturonic acid sodium salt (SIGMA).

### Amylase:

One unit of activity corresponded to the quantity of enzyme releasing lumol of maltose reducing sugar (in glucose equivalents) per minute per ml under the assay conditions. The substrate used herein for assessing the activity in the current invention is Potato starch soluble (HIMEDIA).

### Mannanase:

One unit of activity corresponded to the quantity of enzyme releasing lumol of mannose reducing sugar (in glucose equivalents) per minute per ml under the assay conditions. The substrate used for assessing the activity in the current invention is Locust bean gum from Ceratonia siliqua seeds (SIGMA).

### Laccase:

1 IU is defined as amount of enzyme required to oxidize 1 micromole of guaiacol per min. The substrate used for assessing the activity in the current invention where the substrate is Guaiacol (SIGMA- ALDRICH).

The term “degumming” as used herein is defined as the process of separating fibres from each other by removing biomolecules such as pectins, starch, hemicellulose, gums and other biomolecules which binds the fibretogether.

The terms ″textile grade fibre″ and ″textile fibre″ are used interchangeably herein, and refer to fibres that are soft and have characteristics that are suitable for making textiles.

Tensile strength and breaking elongation are two of the most important mechanical properties for a textile grade fibre. Fibre tensile streng this of ten expressed by tenacity with a unit of force per denier ortex.

The term “cellulosic fibre” as used herein is defined as fibres comprising at least 20 % of cellulose, and are made with ethers or esters of cellulose, which are of plant origin and can be obtained from the bark, wood or leaves of plants, or from other plant parts. In addition to cellulose, the fibres may also contain other components such pectins, hemi cellulose, lignin as majorly apart from other minor constituents. With different sources percentages of these components vary altering the mechanical properties of the fibres.

The term “man-made fibres” as used herein is defined as fibres that do not exist in nature, and are usually made from various chemicals, or are regenerated from plant fibres. Examples of man-made fibres, include polyester; polyamide - (nylon); acrylics; viscose, regenerated cellulosic fibres such as rayon, bamboo fibre, Lyocell, Modal, diacetate fibre, triacetate fibre, but are not limited to these.

The term “treatment bath” or “bath” as used herein is the liquid/ medium in which the enzymatic treatment is done.

The term “Denier”, as used herein, is used to describe the fineness of a textile material that is quantified as the materials weight in grams per 9,000 meters of that material.

The term elongation is defined as s a percentage of the starting length. The elastic elongation is of decisive importance since textile products without elasticity would hardly be useable. They must be able to deform and also return to shape. Higher elongation of textile fibresleads to easier processing and also increase comfort duringwear.

The term “degree of reflectance” of a fibre, as used herein, is defined as the measure of the fraction of light that is reflected by a material or its reflectance.

The term “breaking strength” of a fibre, as used herein, is defined as the maximum amountof tensile stress that the material can withstand before breaking ordeformation. A loom is a device for weaving threads for getting cloth. A loom produces fabric by interlacing a series of lengthwise, parallel yarns width a series of width wise parallelyarns.

### Handloom:

A hand loom is a simple machine, powered by hand, and used for weaving.

A power loom is a type of loom that is powered mechanically instead of using human power to weave patterns or thread into cloth.

### Embodiments

One embodiment of the current invention is an enzyme formulation for converting raw natural fibres to textile grade fibres comprising enzymes selected from the group consisting of Cellulase, xylanase, pectinase, polygalacturonase, lipase, alpha amylase, mannanase and laccase.

In one embodiment, the enzyme formulation disclosed herein comprises 800-1000 U/ml Cellulase, 10,000-15000 U/ml xylanase, 100-300 U/ml pectinase, 100-200 U/ml polygalacturonase, 500-700 U/ml lipase, 300-500 U/ml alpha amylase, 50-100 U/ml mannanase, 10-20 U/ml laccase or a combination thereof.

In one embodiment, the enzyme formulation is added at a concentration of 0.5-1% to the fibre treatment bath for conversion of raw natural fibres to textile grade fibres.

In one embodiment, the enzyme formulation is stable at pH 4.5-5.5 and at temperature range of 20-40° C.

In one embodiment, the enzyme formulation further comprises non-ionic surfactant and at least one stabilizer.

In one embodiment, the enzyme formulation comprises a stabilizer and the stabilizer is propylene glycol, glycerol, sugar or sugar alcohol.

In one embodiment, the textile grade fibres produced from raw natural fibres by treating with the enzyme formulation disclosed herein,are spinnable into yarn.

In one embodiment, the yarn produced from the textile grade fibres produced by using the enzyme formulation disclosed herein is woven by handloom or powerloom.

In one embodiment, the enzyme formulation converts raw fibres that have not been pretreated by acid or alkali into textile grade fibres.

In one embodiment, the raw natural fibres are from banana, hemp, nettle, flex, jute, pineapple sisal, or remi plants.

In one embodiment, the textile grade fibres are maximum 75 dpf and breaking strength is at least 50 g.

A method of making textile grade fibres from raw natural fibres, the method comprising the step of contacting the raw natural fibres with the enzyme formulation disclosed herein for 2-4 hours, wherein the enzyme formulation is present at a concentration of 0.5 - 1% in the fibre treatment bath.

In one embodiment, the concentration and combination of each enzyme depends on composition of raw fibres.

In one embodiment, the enzymatic formulation removes the biomolecules holding the cellulosic fibres together in plant derived biomass. Examples of such biomolecules includes, but is not limited to, pectins, gums, starch, and xylans

In one embodiment, removal of these biomolecules from the cellulosic fibres using the enzymatic formulation disclosed herein makes fibres softer and brighter, compared to raw natural fibres, or fibres extracted from raw natural fibres by conventional means.

In one embodiment, Pectinase releases individual fibres from fibre bundles. In one embodiment, Xylanase increases brightness index of the fibres.

In one embodiment, treatment with the enzymatic formulation enhances fibre quality by increasing parameters such as good tensile strength, brightness index, flexibility and fibre fineness required in textile industry.

Formulation of enzyme plays important role both in achieving better results at applicationand stability front.

In one embodiment, the enzymatic formulation contains salts, examples of which include, but are not limited to, sodium and magnesium salts.

In one embodiment, the salts increase stability, enzyme activity and its effectiveness in fibre processing. In one embodiment, bio-compatible surfactant such as alpha olefin sulfonate (AOS) is added to the enzyme formulation for improved removal of surface impurities.

In one embodiment, the enzymatic formulation disclosed herein is stable at the pH range between3-8.

In one embodiment, the enzymatic formulation disclosed herein is stable at the pH range between5-6.

In one embodiment, the enzymatic formulation disclosed herein is stable at the temperature range between 40-70° C.

In one embodiment, the enzymatic formulation disclosed herein is stable at the temperature range of 45- 60° C.

In one embodiment, the enzymatic formulation disclosed herein acts at pH 5 to convert raw natural fibres to textile gradefibres.

In one embodiment, the enzymatic formulation disclosed herein acts at 50° C. to convert raw natural fibres to textile gradefibres.

In one embodiment, the combination and concentration of enzyme depends on source of raw material.

In one embodiment, the second enzymatic formulation improves the fineness of the fibre without affecting the strength of the fibre for making yarn from the fibres by automated methods.

In one embodiment, the enzymatic formulations disclosed herein act to produce textile grade fibres which are suitable for spinning yarns of different blends for textile applications. In one embodiment, the treatment of the raw natural fibres with the enzymatic formulation is followed by a hot wash step, or a neutralization step.

In one embodiment, the plant derived biomass is from plants, examples of which include, but are not limited to, banana, hemp, jute, nettle, flex, bamboo, pineapple, sisal and remi.

In one embodiment, the plant derived biomass used for the current invention is stem, leaves. In one embodiment stems from banana, ramie, bamboo are used as plant derived biomass.

In one embodiment, the raw natural fibres are cellulose based fibres, wherein they comprise at least 20% of cellulose. In one embodiment, the raw natural fibres also comprise lignin, hemicellulose, pectins, xylans, mannans but not limited to these.

In one embodiment, the plant derived biomass may be agricultural waste products suchas banana pseudostem, hemp stalk, Flex stalk, Jute cotton stems but not limited to these.

In one embodiment, the enzymatic formulation disclosed herein is used for converting banana, hemp or jute fibres into textile grade fibres.

In one embodiment, the textile grade fibres produced by the activity of the enzymatic formulations disclosed herein have high tensile strength, good elongation, better spinnability, high tenacity, low resistance, finer diameter and high brightness index compared to the fibres obtained by chemical processing of raw natural fibres.

In one embodiment, the fibre diameter of the textile grade fibre produced by the activity of the enzymatic formulations disclosed herein, is not more than 75 denier.

In one embodiment, the breaking strength of the textile grade fibre is not less than 50 g.

In one embodiment, the tenacity or the strength of the fibre produced by the activity of the enzymatic formulations described herein is not less than 1.2 grams/denier.

In one embodiment, the tenacity or the strength of the fibre produced by the activity of the enzymatic formulations described herein is not less than 1.3 grams/denier for fibre produced from hemp.

In one embodiment, the tenacity or the strength of the fibre produced by the activity of the enzymatic formulations described herein is not less than 2.5 grams/denier for fibre produced from banana

In one embodiment, the elongation of the fibre produced by the activity of the enzymatic formulations described herein is not less than 5 %.

In one embodiment, the brightness (degree of reflectance) is not less than 65.

In one embodiment, the textile grade fibres produced by the activity of the enzymatic formulations disclosed herein are used for producing any material including, but not limited to, apparel, suiting - shirting, upholstery, handicrafts, doll hairs, andfootwear.

In one embodiment, power loom is used for weaving fabric from the yarn spun from the textile grade fibres produced by the activity of the enzymatic formulation disclosedherein.

In one embodiment, the fibres produced using the enzyme formulation disclosed herein are spun into yarns using automatic machines/ process which can be further woven into fabrics using handloom or powerlooms

In one embodiment, handloomisusedfor weaving fabrics from the yarn spun from the textile grade fibres produced by the activity of the enzymatic formulation disclosedherein.

In one embodiment, the textile grade fibres produced by the activity of the enzymatic formulations disclosed herein, are further woven or knitted.

In one embodiment, the textile grade fibres produced by the activity of the enzymatic formulations disclosed herein are further blended with other natural or man-made fibres, examples of which include, but are not limited to, regenerated cellulosic fibres, linen, cotton, and manmade fibres.

In one embodiment, treatment with the disclosed enzyme formulation leads to complete removal of pectin and at least 40% decrease in lignin content from the raw natural fibres to make to textile grade fibres.

### EXAMPLES

### Materials and Methods

The formulation designed and tested contained multiple enzymes such as pectinase (100-1000 U/ml), polygalacturonase (100-1000 U/ml), xylanase (7000-15000), cellulase (500-2000 U/ml), lipase (500-1000 U/ml), alpha amylase (100-500 U/ml), mannanase (50-200 U/ml) or laccase (5-20 U/ml).

The enzyme activities were assayed as given in the detailed description (references 1-6)

The sources of the enzymes used for the current experiments were:
Cellulase/xylanase :Trichodermareesei
Pectinase, polygalaturonase and lipase: Aspergillus niger
Mannnanase: Bacillus sp.
Laccase: fungal source with min activity of 25 U/ml.

Formulation also included anionic salts such as chloride, Sulphate, nitrate, phosphate, carbonate, or combinations thereof in the concentration of 2-5% of final formulation (w/v). Formulation also included non-ionic surfactant ssuch as Polysorbates, Tri Decyl Alcohol Ethoxylates, Sorbitan Esters, ethoxylated and alkoxylated fatty acids, ethoxylated amines, ethoxylated alcohol, alkyl or nonyl-phenol ethoxylates in the concentration of 1-5%. Formulation also included stabilizers such as e.g., a polyol such as propylene glycol orglycerol, a Sugar or Sugar alcohol in the concentration ranging 5-10%

Formulation also included colorants in the concentration 0.01-0.1%. pH of the formulation ranged between 4.5 to 5.3

Formulation was found to be maximally active between temperature 30-60° C. and pH range 3-7 and showing optima at 40° C. and pH 5.

The process involving enzymatic treatment showed effective degumming by almost complete removal of pectins, lignin in the range of 40-75% after treatment compared to untreated samples.

Treatment with this formulation resulted in enhancing cellulosic content in the treated biomass by 30-50% compared to untreated samples.

### Example 1: Making Textile Grade FibresFrom Banana Fibre

Objective:the objective of the invention was to overcome the problems for banana fibre degumming, softening, and improving fineness in finished fibres with the help of cocktail enzymes formulation to prepare the method for banana fibre for processing.

### Experiment 1:

Formulation 1: Formulation 1 was prepared by adding following constituents in distilled water in a step wise manner.

First enzymes such as Cellulase 250-300 U/ml, xylanase: 750-800 U/ml, pectinase: 15-20 U/ml, polygalacturonase: (35-40 U/ml) and laccase (1 U/ml), was added in distilled water. Further Anionic salts Sodium chloride, sulphates combinedly at conc. of 2.5-4.0% were added, followed by non-ionic surfactant: tridecyl alcohol etholxylates 2-3%.

Formulation was stabilized by adding stabilizer glycerol (5-7%) and colorant-(0.01%). Concentration indicated are the final concentrations of the components in the formulation.

### Results:

Formulation 1 when used in banana fibre processing trial, there was no improvement in fineness and softness in final product. So, decided to improve dosages of enzymes in next trial.

Formulation 2: Formulation was prepared by adding following constituents in distilled water in a step wise manner.

First enzymes such as Cellulase 2700-2800 U/ml, xylanase: 17500-18000 U/ml, pectinase: 350-450 U/ml, polygalacturonase: (10-15 U/ml) were added in distilled water.

Further anionic salts Sodium chloride, sulphates combinedly at conc. of 2.5-4.0% were added, followed by non-ionic surfactant: tridecyl alcohol etholxylates 2%.

Formulation 2 was stabilized by adding stabilizer glycerol (5-7%) and colorant-(0.01%). Concentrations indicated are the final concentrations of the components in the formulation.

### Results:

When Formulation 2 was used in banana fibre processing trial, it was observed that improved fibrequality but weight loss was found to be maximum upto 40% after processing.

So, we decided to reduce the concentration of enzymes in the formulation in next trial (formulation 3).

Formulation 3: Formulation 3 was prepared by adding following constituents in distilled water in a step wise manner.
a. As a first step, enzymes such as Cellulase (EC 3.2.1.4, 1,4-β-D-glucan 4-glucanohydrolase) having endocellulase activity 800-1000 U/ml and, xylanase (EC 3.2.1.4, Endo-1, 4-β -xylanase): 10,000-15000 U/ml., pectinase: 100-300 U/ml, polygalacturonase (EC.3.2.1.15, poly-alpha-1,4-galacturonide glycanohydrolase): (100-200 U/ml lipase (EC 3.1.1.3, Triacylglycerol acylhydrolase) (500-700 U/ml), alpha amylase (EC 3.2.1.1, 1,4-alpha-D-glucan glucanohydrolase) (300-500 U/ml), β-1,4-mannanase (50-100 U/ml) and laccase (10-20 U/ml), were added in distilled water.
b. Further Anionic salts Sodium chloride, sulphates at conc. of 1-2% were added, followed by non-ionic surfactant: tridecyl alcohol etholxylates 3-5%,
c. Formulation was stabilized by adding stabilizer glycerol (7-10%) and colorant-(0.01%).
d. Concentrations indicated are the final concentrations of the components in the formulation.

### Fiber Treatment Methodology

a. Dried banana fibres were treated with enzyme cocktail formulation atdosing of 5 gpldepending upon the material liquor ratio (MLR) of 1:10.
b. Enzymatic treatment was given for 2 hours at optimum temp of 40° C. and pH 5
c. Fibers were than bleached in 0.5 % sodium hydroxide and 0.7 % hydrogen peroxide solution for 1 hour and dried
d. Fiber was further opened using mechanical process such as combing.

### Results From Formulation 3 Treatments:

Fibre parameter of treated and untreated samples:

| Sample particulars | Fiber Denier ASTM D 1577 | Breaking strength (g) | Breaking extension (%) | Calculated tenacity (g/d) |
|---|---|---|---|---|
| Untreated fibres | 102.0 | 505.55 | 9.33 | 4.96 |
| Treated fibres | 57.95 | 119.50 | 19.41 | 2.06 |

Composition analysis of treated and untreated biomass:

| Samples | Pectin (%) | Cellulose (%) | Lignin+Ash (%) |
|---|---|---|---|
| Untreated Fiber | 2.02 ± 0.25 | 45.95 ± 0.5 | 20.93 ± 5.47 |
| Treated fibres | 0^{∗} | 64.06 ± 1.51 | 11.87 ± 0.210 |

^{∗} Since peak was not detected in HPLC after treatment

### CONCLUSION

After treatment with formulation 3, Fibre diameter reduced by 40-50% in treated sample compared to untreated samples. The treated fibres showed complete removal of pectins and reduction of lignin by 43.3%

### Example 2

### Raw Fibre: Hemp

The present example describes processing of natural bio-fibre such as Hemp fibre using an enzyme composition.

The process was performed to overcome the problems for hemp fibre degumming, softening and fineness improvement with the help of cocktail enzymes formulation to prepare the method for hemp fibre for processing.

### Formulation:

Formulation was prepared by:
a. Addingcellulase (EC 3.2.1.4, 1,4-β-D-glucan 4-glucanohydrolase)400-600 U/ml, xylanase (EC 3.2.1.4, Endo-1, 4-β -xylanase):7000-10000 U/ml, pectinase: 300-500 U/ml, polygalacturonase (EC 3.2.1.15, poly-alpha-1,4-galacturonide glycanohydrolase): (200-400 U/ml), lipase (EC 3.1.1.3, Triacylglycerol acylhydrolase) ) (500-1000 U/ml) and laccase (10-20 U/ml) in distilled water,
b. Further anionic salts Sodium chloride was added at concentration of 2% (w/v),
c. Non-ionic surfactant, tridecyl alcohol etholxylates was then added at 3% (w/v), Formulation was stabilized by adding stabilizer: glycerol at 10% and colorant-0.01%
d. Volume was made up using Distilled water.

Fibre treatment Methodology: hemp fibre provided by the invention, to overcome the degumming cleaning, softening and fineness improvement to prepare the method for hemp fibre, comprise the steps:

a. Dried hemp fibres were treated with enzyme cocktail formulation atdosing of 5 gpl depending upon the material liquor ratio (MLR) of 1:10.

b. Enzymatic treatment was given for 2 hours at temp of 40° C. and at pH 5.

c. Enzymatic treatment was completed after rinsing the fibres.

d. Fibers were than bleached (5gpl Sodium hydroxide/5gpl Hydrogen peroxide solution) and dried.

e. Fiber was further opened using mechanical process such as combing.

### Results:

Fibre parameter of treated and untreated samples:

| Sample particulars | Fiber Denier ASTM D 1577 | Breaking strength (g) | Breaking extension (%) | Calculated tenacity (g/d) |
|---|---|---|---|---|
| Untreated fibres | 156.70 | Not performed# | Not performed# | Not performed# |
| Treated fibres (formulation 3) | 43.84 | 57.12 | 7.12 | 1.30 |

#Due to coarseness of the raw fibres

Compositional analysis of treated and untreated hemp samples:

| Samples | Pectin (%) | Cellulose (%) | Lignin+Ash (%) |
|---|---|---|---|
| Raw Hemp | 3.68 ± 0.06 | 50.05 ± 2.18 | 12.59 ± 2.14 |
| Treated Hemp (formulation 3) | 0^{∗} | 89.82 ± 1.91 | 6.81 ± 1.46 |

^{∗} Since peak was not detected in HPLC after treatment

### CONCLUSION

Fiber diameter reduced by more than 70% in treated sample compared to untreated samples. The treated fibres showed complete removal of pectins and reduction of lignin by 46%.

Treatment with Formulation 3 showed the greatest improvement in fibre properties of the raw natural fibres.

### REFERENCES

1. T.K Ghosh, measurement of cellulase activities, Pure &App!. Chem., Vol. 59, No. 2, pp. 257-268, 1987
2. Bailey, M.J., Biely, P. and Poutanen, K. (1992) Interlaboratory testing of methods for assay of xylanase activity. J. Biotechnol. 23, 257-270.
3. Biz A, Farias FC, Motter FA, de Paula DH, Richard P, Krieger N, et al. (2014) Pectinase Activity Determination: An Early Deceleration in the Release of Reducing Sugars Throws a Spanner in the Works! PLoS ONE 9(10): e109529. https://doi.org/10.1371/journal.pone.0109529
4. Alexander V. Gusakov, Elena G. Kondratyeva, Arkady P. Sinitsyn, “Comparison of Two Methods for Assaying Reducing Sugars in the Determination of Carbohydrase Activities”, International Journal of Analytical Chemistry, vol. 2011, Article ID 283658, 4 pages, 2011. https://doi.org/10.1155/2011/283658.
5. Miller G. L. Use of Dinitrosalicylic acid regent for determination of reducing sugars. Analytical Chemistry 31(3) 426-428 1959
6. Rehan A. Abd El Monssef, Enas A. Hassan, Elshahat M. Ramadan, “Production of laccase enzyme for their potential application to decolorize fungal pigments on aging paper and parchment, Annals of Agricultural Sciences, Volume 61, Issue 1, Pages 145-154,2016,

We claim:

1. An enzyme formulation for converting raw natural fibres to textile grade fibres comprising enzymes selected from the group consisting of Cellulase, xylanase, pectinase, polygalacturonase, lipase, alpha amylase, mannanase and laccase.

2. The enzyme formulation of claim 1, wherein it comprises 800-1000 U/ml Cellulase, 10,000-15000 U/ml xylanase, 100-300 U/ml pectinase, 100-200 U/ml, polygalacturonase, 500-700 U/ml lipase, 300-500 U/ml alpha amylase, 50-100 U/ml mannanase and 10-20 U/ml laccase .

3. The enzyme formulation of claim 1, wherein it is added at a concentration of 0.5-1% to the fibre treatment bath for conversion of raw natural fibres to textile grade fibres.

4. The enzyme formulation of claim 1, wherein it is stable at pH 4.5-5.5 and at temperature range of 20-50° C.

5. The enzyme formulation of claim 1, wherein it further comprises non-ionic surfactant and at least one stabilizer.

6. The enzyme formulation of claim 4, wherein the stabilizer is propylene glycol, glycerol, sugar or sugar alcohol.

7. The enzyme formulation of claim 1, wherein the textile grade fibres are spinnable into yarn.

8. The enzyme formulation of claim 7, wherein the yarn produced from the textile grade fibres is woven by handloom or powerloom.

9. The enzyme formulation of claim 1, wherein it converts raw fibres that have not been pre-treated by acid or alkali into textile grade fibres.

10. The enzyme formulation of claim 1, wherein the raw natural fibres are from banana, hemp, nettle, flex, jute, or remi plants.

11. The enzyme formulation of claim 1, wherein the textile grade fibres are maximum 75 dpf and breaking strength is at least 50 g.

12. A method of making textile grade fibre from raw natural fibres, the method comprising the step of contacting the raw natural fibres with the enzyme formulation of claim 1 for 2-4 hours, wherein the enzyme formulation is present at a concentration of 0.5 - 1% in the fibre treatment bath.
